(19) United States
(12) Patent Application Publication
(10) Pub. No.: US 2020/0239378 A1
(43) Pub. Date: Jul. 30, 2020

(54) DEVICE FOR ELECTROCHEMICALLY ASSISTED AEROBIC COMPOSTING OF ORGANIC SOLID WASTE AND METHOD THEREFOR

(71) Applicant: FUJIAN AGRICULTURE AND FORESTRY UNIVERSITY, Fuzhou (CN)

(72) Inventors: Shungui ZHOU, Fuzhou (CN); Jiahuan TANG, Fuzhou (CN); Xiang LI, Fuzhou (CN)

(21) Appl. No.: 16/826,199

(22) Filed: Mar. 21, 2020

Related U.S. Application Data
(63) Continuation of application No. PCT/CN2018/093205, filed on Jun. 28, 2018.

(30) Foreign Application Priority Data
Apr. 2, 2018 (CN) 201810281655.9

(51) Int. Cl. C05F 17/30; C12M 1/42; C12N 13/00; C05F 3/06; C05F 7/00

(52) U.S. Cl. CPC C05F 17/30; C12M 35/02; C05F 7/00; C05F 3/06; C12N 13/00

(57) ABSTRACT

The present disclosure discloses a device for electrochemically assisted aerobic composting of organic solid waste and method therefor. The aerobic composting device includes a compost bioreactor, a working electrode, an auxiliary electrode, and a reference electrode. The working electrode is situated on the inner wall of the compost bioreactor, and the reference electrode is situated between the working electrode and the auxiliary electrode. The bottom of the compost bioreactor is provided with a ventilation pipeline with an aeration head. A method for electrochemically assisted aerobic composting of organic solid waste by using the device is also disclosed. By performing electrochemically assisted composting through the composting device of the present disclosure can enrich and utilize electric energy microorganisms to promote the progress of the redox reaction in the compost pile, quickly increase the temperature of the compost, shorten the compost period, and increase the compost maturity.

### CROSS REFERENCE OF RELATED APPLICATIONS

This application claims all benefits accruing under 35 U.S.C. § 119 from China Patent Application No. 201810281655.9, entitled “DEVICE FOR ELECTROCHEMICALLY ASSISTED AEROBIC COMPOSTING OF ORGANIC SOLID WASTE AND METHOD THEREFOR” filed on Apr. 2, 2018, in the State Intellectual Property Office of China, the content of which is hereby incorporated by reference. This application is a continuation under 35 U.S.C. § 120 of international patent application PCT/CN2018/093205 filed on Jun. 28, 2018, the content of which is also hereby incorporated by reference.

### TECHNICAL FIELD

The present disclosure relates to a device for electrochemically assisted aerobic composting of organic solid waste and a method therefor.

### BACKGROUND

The production of organic solid waste in China has the characteristics of large quantity, wide area, wide variety, and complex nature. At present, the annual quantities of livestock manure sludge, crop straws, domestic garbage, and urban sludge in China has reached about 3 billion tons, 900 million tons, 180 million tons, and 40 million tons, respectively. Dealing with these increasingly large quantities of organic solid waste generated every day has become a serious social problem. Transforming the organic solid waste into the available resources and energy for human beings according to the local conditions and realizing its harmless and resource recycling is a major practical problem in the sustainable development of the society, which needs to be solved urgently.

The current organic solid waste disposal methods in China include sanitary landfill, incineration, anaerobic digestion, and high-temperature aerobic fermentation. Wherein the conversion of organic solid waste into organic fertilizer through high-temperature aerobic fermentation technology not only effectively solves the problem of environmental pollution of organic waste but also develops the organic fertilizer industry, improves soil fertility and maintains sustainable agricultural development.

High-temperature aerobic fermentation (also known as aerobic composting) is an important technology for organic solid waste recycling. The basic principle is that under aerobic conditions, biodegradable organic matter is converted into stable humus by the action of microorganisms and the generated high temperature kills pathogenic bacteria and worm eggs so that the organic waste is stabilized. The advantages of this method are low investment cost, resource utilization of subsequent products, and suitability for large-scale promotion. In conventional high-temperature aerobic composting, the optimal growth temperature of most thermophilic microorganisms is about 60° C., where conventional composting uses low compost temperature, long composting period, incomplete killing of pathogenic microorganisms, and poor maturation effects (poor quality of fermentation follow-up products). Moreover, the high-temperature aerobic fermentation process requires a large amount of long-term aeration. Since oxygen is difficult to dissolve in water, most oxygen is directly lost without microbial utilization, which results in a large amount of ineffective aeration, which increases energy consumption cost, odor diffusion, and loss of ammonia nitrogen, thereby restricting the popularization and application of composting technology.

The essence of aerobic composting technology is: the microbial flora in the compost pile biologically oxidizes organic matter (partially oxidized to a more stable organic matter, or completely mineralized into carbon dioxide and water) to generate electrons to transfer to the electron acceptor, namely oxygen. The science essence is the electron transfer reaction between an organic matter as an electron donor and oxygen as an electron acceptor. With the help of electrochemical technology, the electrons generated by the oxidation of organic matter by microorganisms can be accelerated transfer to the electrode, thereby accelerating the composting process. It does not require aeration but can achieve similar effects as oxygen. CN106946601A discloses a method for fermenting and composting livestock and poultry manure. The method realizes the removal of antibiotics and the recovery of heavy metals in livestock and poultry manure by adding a direct current of 0.5-3.0 V/cm to the composting system. At the same time, it accelerates the composting reaction process to achieve improved composting efficiency and a shortened composting cycle. However, this technical solution provides high direct current, and excessive voltage leads to water electrolysis, which is not suitable for the survival of electricity-producing microorganisms. Also, in this solution, it is necessary to add a composite catalyst with a mass ratio of 0.05-2%. The preparation process of the composite catalyst is relatively complicated and must proceed through pollution processes like calcination, but there are problems with this technique, such as secondary pollution. CN104671863A discloses a bioelectrochemically-assisted anaerobic composting device and a method for starting the device to improve the maturity of dewatered sludge. The technical solution is to introduce bioelectrochemical technology into the sludge anaerobic fermentation, and after the sludge undergoes anaerobic fermentation for 35 to 40 days, its maturity is increased by 30%. However, the system is anaerobic fermentation, and the fermentation temperature is still low (30-45° C.). In this temperature range, various pathogenic bacteria and eggs are not completely killed, and the quality of the subsequent fermentation products is poor. Moreover, the system is divided into an anaerobic composting room and an electrochemical room, and the system configuration is complicated. In addition, the start-up process of the system is cumbersome. Electrochemically active bacteria must be acclimated in the early stage, which requires changes to the electrolyte every 3-5 days. After several cycles, the culture can be completed, and the system can be considered to be started.

### SUMMARY

The object of the present disclosure is to provide an electrochemically assisted device and method for aerobic composting of organic solid waste. The device can achieve rapid aerobic composting of organic solid waste, increase the temperature of the compost, and accelerate the process under low aeration conditions. At the same time, production costs are saved.

The basic principle of the present disclosure is shown as below:

In the aerobic composting of organic solid waste, the working electrode and auxiliary electrode are arranged to provide a certain potential (a low direct voltage, such as 2 V or 5 V) to the working electrode, so as to form an electric field between the working electrode and the auxiliary electrode. Under the stimulation of the electric field, a large number of electric energy microorganisms are enriched. The electric energy microorganisms can use the electrodes as the final electron receptor of the respiratory chain for propagation and metabolism. The advantage of electric energy microorganisms is that propagation and metabolism are not limited by the concentration of dissolved oxygen in the compost pile, so much aeration is saved. At the same time, the dominant microorganisms in the conventional compost are not affected by the electric field but continue to consume organic matter to generate heat. Under the action of the above microorganisms, the temperature of the compost can be rapidly increased, so as to improve the killing efficiency of various pathogenic bacteria and eggs and improve the humification degree of compost products.

The technical solution adopted by the present disclosure is as follows:

A device for electrochemically assisted aerobic composting of organic solid waste, including a compost bioreactor, a working electrode, an auxiliary electrode, and a reference electrode; the working electrode is provided on an inner wall of the compost bioreactor, and the reference electrode is situated between the working electrode and the auxiliary electrode; a ventilation pipeline with an aeration head is provided at a bottom of the compost bioreactor.

In the device, the distance between the working electrode and the auxiliary electrode is 10 cm to 100 cm.

In the device, the working electrode is at least one selected from the group consisting of a stainless steel plate, a graphite plate, a carbon felt, and a carbon cloth electrode; the auxiliary electrode is at least one selected from the group consisting of a carbon felt, a carbon cloth, and a graphite electrode; the reference electrode is one selected from the group consisting of a Hg/HgO electrode, an Ag/AgCl electrode, a hydrogen standard electrode, and a saturated calomel electrode.

In the device, the potential of the working electrode relative to the reference electrode is set to −5 V to 5 V.

In the device, the working parameters of the aeration of the ventilation pipeline are: once to twice a day, each aeration is for 15 minutes to 30 minutes, the flow rate of the aeration is 0.1 L/min·m³ to 0.2 L/min·m³.

In the device, the compost bioreactor is covered with a thermal insulation layer.

A method for electrochemically assisted aerobic composting of organic solid waste, including the following steps:

1) mixing organic solid waste and auxiliary material, adding water and stirring evenly to obtain compost raw material;

2) placing the composting raw material into the device mentioned above to perform composting.

In step 1), the mass ratio of the organic solid waste and the auxiliary material is 10:(1 to 3).

In step 1), the water content of the compost raw material is 50 wt % to 60 wt %.

The advantageous effects of the present disclosure are shown as below:

The present disclosure discloses a novel electrochemically assisted composting device and applies it to aerobic composting of organic solid waste. Performing electrochemically assisted composting through the composting device provided by the present disclosure can enrich and utilize electric energy microorganisms, promote the progress of the redox reaction in the compost pile, quickly increase the temperature of the compost, shorten the compost period, and increase the compost maturity. The present disclosure overcomes the defects of conventional aerobic compost with a large number of ineffective aeration methods and wasted energy consumption, has advantages in energy saving, simple equipment maintenance, high processing efficiency, and stable operation, and can be widely used in resource recycling and safe disposal of organic solids, such as urban sludge, livestock and poultry manure, crop straw, and domestic garbage.

### BRIEF DESCRIPTION OF THE DRAWINGS

FIG. 1 is a schematic structural diagram of a device provided by the present disclosure;

FIG. 2 is a graph showing changes in compost temperature with the turning compost time in embodiment 1 and comparative example 1;

FIG. 3 is a graph showing changes in compost temperature with the turning compost time in embodiment 2 and comparative example 2;

FIG. 4 is a graph showing changes in compost temperature with the turning compost time in embodiment 3 and comparative example 3.

### DETAILED DESCRIPTION OF THE EMBODIMENTS

A device for electrochemically assisted aerobic composting of organic solid waste, including a compost bioreactor, a working electrode, an auxiliary electrode, and a reference electrode. The working electrode is provided on the inner wall of the compost bioreactor, and the reference electrode is situated between the working electrode and the auxiliary electrode. A ventilation pipeline with an aeration head is provided at the bottom of the compost bioreactor.

Preferably, in the device, the compost bioreactor is a cylindrical reactor or a strip reactor.

Further, when the compost bioreactor is a cylindrical reactor, the diameter is 50 cm to 80 cm, and the height is 80 cm to 120 cm; when the compost bioreactor is a strip reactor, the length is 1.5 m to 2.5 m, the width is 0.5 m to 1.5 m, and the height is 0.5 m to 1.5 m.

Further, in the device, the distance between the working electrode and the auxiliary electrode is 10 cm to 100 cm.

Preferably, in the device, the working electrode is at least one selected from the group consisting of a stainless steel plate, a graphite plate, a carbon felt, and a carbon cloth electrode; the auxiliary electrode is at least one selected from the group consisting of a carbon felt, a carbon cloth, and a graphite electrode; the reference electrode is one selected from the group consisting of a Hg/HgO electrode, an Ag/AgCl electrode, a hydrogen standard electrode, and a saturated calomel electrode.

Preferably, in the device, the potential of the working electrode relative to the reference electrode is set −5 V to 5 V.

Preferably, in the device, the diameter of the ventilation pipeline is 8 mm to 20 mm.

Preferably, in the device, working parameters of the aeration of the ventilation pipeline are: once to twice a day, each aeration is for 15 minutes to 30 minutes, and the flow rate of the aeration is 0.1 L/min·m³ to 0.2 L/min·m³.

Preferably, in the device, the compost bioreactor is covered with a thermal insulation layer. Further, the thermal insulation material used for the thermal insulation layer is a conventional choice in the field and can be one selected from the group consisting of quilts, aluminum foil self-adhesive rubber-plastic sheets, high-temperature resistant aluminum silicate needle punched ceramic fiber thermal insulation cotton, flame-retardant rubber-plastic sponge, and glass wool.

Preferably, in the device, a temperature probe (online thermometer) is set around the compost bioreactor to record the temperature change of compost in real-time.

A method for electrochemically assisted aerobic composting of organic solid waste, including the following steps:

1) mixing organic solid waste and auxiliary material, adding water and stirring evenly to obtain compost raw material;

2) placing the composting raw material into the device for electrochemically assisted aerobic composting of organic solid waste mentioned above to perform composting.

Preferably, in step 1), the mass ratio of the organic solid waste and the auxiliary material is 10:(1 to 3).

Preferably, in step 1) of the composting method, the organic solid waste is at least one of chicken manure, duck manure, goose manure, pig manure, cow manure, dog manure, cat manure, sheep manure, horse manure, or sludge.

Preferably, in step 1) of the composting method, the auxiliary material is at least one of rice bran, straw, or rice husk.

Preferably, in step 1), the water content of the compost raw material is: 50 wt % to 60 wt %.

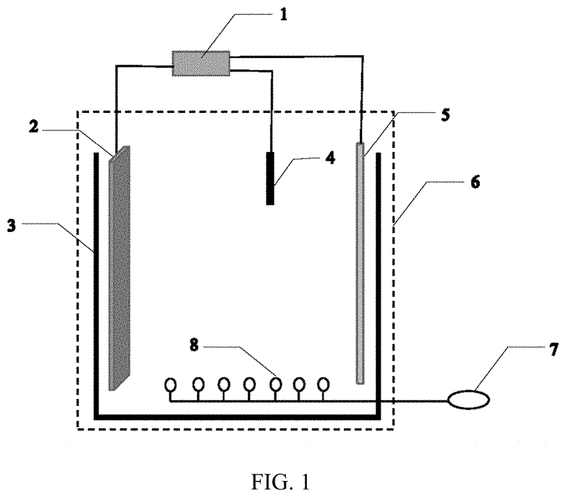

FIG. 1 is a structural diagram of the device of the present disclosure, which only represents one example of the device of the present disclosure, and the device of the present disclosure is not limited to the structure shown in the diagram. In FIG. 1, 1—potentiostat; 2—working electrode; 3—the outer wall of compost bioreactor; 4—reference electrode; 5—auxiliary electrode; 6—thermal insulation layer; 7—air pump; 8—aeration head.

In combination with FIG. 1, the content of the present disclosure will be further described in detail through specific embodiments. The raw materials used in the embodiment can be obtained from conventional commercial channels without special description.

### Embodiment 1

1) Design of Compost Bioreactor:

When the cylindrical compost reactor is adopted, the diameter is 55 cm and the height is 100 cm. A rectangular working electrode is arranged along the inner wall of the plastic barrel, which is made of stainless steel sheet with an area of 3 to 5 m². A cylindrical auxiliary electrode is placed in the center of the plastic barrel, the circular tube is the inner center with a diameter of 5 cm and a height of 8 0 cm, and a saturated calomel reference electrode is connected at the upper portion of the plastic barrel. The bottom of the plastic barrel is provided with a ventilation pipeline, wherein the diameter of the ventilation pipeline is 10 mm. To ensure uniform aeration, the height of the ventilation pipeline is 30 to 50 cm, from the bottom to the 30 cm position, the aeration holes are punched every 10 cm, and from the 30 cm position to the 50 cm position, and aeration holes are punched every 20 cm. The outer wall of the plastic barrel is wrapped with cotton fiber, which is used for heat preservation of the reactor. At the same time, an online thermometer is set around the plastic barrel to record the temperature change of compost in real-time.

2) Compost Raw Material and Proportion Thereof:

Chicken manure is used as the main compost material and rice bran as the auxiliary material. The above raw material and ingredients are evenly mixed to ensure moisture content at 50% to 60%; the evenly stirred manure is placed into the designed compost bioreactor, connecting the potentiostat, and conducting aerobic composting.

3) Operational Parameters:

Composting at room temperature. Connecting the potentiostat, the potential of the working electrode is designed as 2 V (the reference electrode is a saturated calomel electrode). A small air pump is used as the air source for aeration once a day, the aeration time is 15 minutes each time, and the aeration flow is 0.15 L/min·m³. Every 10 days is the turning cycle at which turning is carried out. When the compost pile is turned over, after mixing evenly, the potentiostat can be connected for recomposting.

### Embodiment 2

When the strip compost reactor is adopted, which is 2 m long, 1 m wide and 1 m high, a rectangular working electrode is arranged along the long side and the material is a graphite plate with an area of about 1 m². On the other side of the long side, an auxiliary electrode of the same size is placed. A saturated calomel reference electrode is connected to the upper portion of the compost pile. The bottom is provided with a ventilation pipeline, wherein the diameter of the ventilation pipeline is 10 mm. To ensure uniform aeration, the height of the ventilation pipeline is 30 to 50 cm, from the bottom to the 30 cm position, aeration holes are punched every 10 cm, and from the 30 cm position to the 50 cm position, the aeration holes are punched every 20 cm. The aeration working parameters are the same as those in Embodiment 1. The outer wall of the reactor is wrapped with cotton fiber for heat preservation of the reactor. At the same time, an online thermometer is set around the interior of the compost pile to record the temperature change of compost in real-time. The potential of the potentiostat is set to +5 V vs SCE (the saturated calomel electrode is the reference electrode).

Sludge is used as the composting raw material, straw is used as the auxiliary material, the mass ratio is 10:3, and the rest is the same as that of Embodiment 1.

### Embodiment 3

In Embodiment 3 and Embodiment 2, the same compost bioreactor is used, pig manure is used as the composting raw material, rice husk as the auxiliary material, a mass ratio of 10:1, and the potential of the potentiostat is set as: −5 V vs SCE (the saturated calomel electrode as reference electrode). The rest is the same as that of Embodiment 1.

### Comparative Example 1

Without using the device for electrochemically assisted aerobic composting of organic solid waste, the same composting raw material as in Embodiment 1 is taken for conventional composting treatment.

### Comparative Example 2

Without using the device for electrochemically assisted aerobic composting of organic solid waste, the same composting raw material as in Embodiment 2 is taken for conventional composting treatment.

### Comparative Example 3

Without using the device for electrochemically assisted aerobic composting of organic solid waste, the same composting raw material as in Embodiment 3 is taken for conventional composting treatment.

### Composting Effect:

### 1. Composting Effects of Embodiment 1 and Comparative Example 1

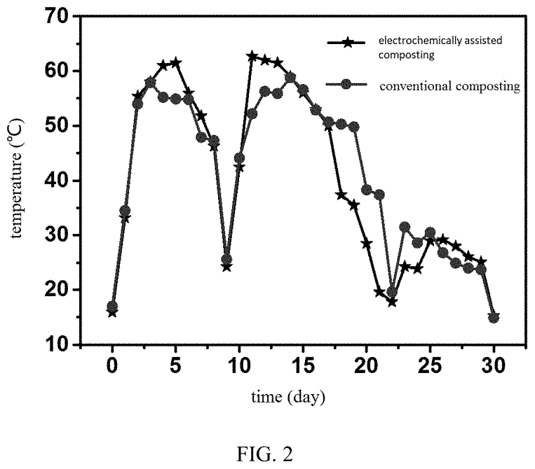

FIG. 2 is a graph showing changes in compost temperature with the turning compost time in embodiment 1 and comparative example 1. As shown in FIG. 2, the maximum temperature of electrochemically assisted composting is 63.5° C., which is 6.2° C. higher than that of conventional composting (57.3° C.). In the second and third cycles of compost turning, the temperature difference is more significant. This shows that the electrochemical method can further improve the compost temperature, which is more conducive to killing eggs, pathogens, etc.

DOM (dissolved organic matter), DON (dissolved organic nitrogen), and EC (electrical conductivity) are the key indicators to characterize the compost maturity. Thus, DOM, DON, and EC of compost were analyzed by three-dimensional fluorescence spectrum after composting for 30 days. The results show that after 30 days composting, the DOM, DON, and EC in the conventional composting system were 855.6 mg/L, 329 mg/L, and 36.2 S/m, respectively, and the removal rate of DOM was 33.8%. The DOM, DON, and EC of the electrochemically assisted composting system were 710.3 mg/L, 469 mg/L, and 30.8 S/m, respectively, and the removal rate of DOM was 45.1%, which is 11.3% higher than that of the conventional composting system. The results show that electrochemically assisted composting promotes the humification of compost.

At the same time, the products from the above two composting methods were tested by verifying the seed germination index. The germination index (GI) of the product seed in conventional compost is 99%; the germination index (GI) of electrochemically assisted compost is 138%, which is 39% higher than that of conventional compost. The results showed that the seed germination rate of compost could be significantly increased by the electrochemically assisted method.

### 2. Composting Effect of Embodiment 2 and Comparative Example 2

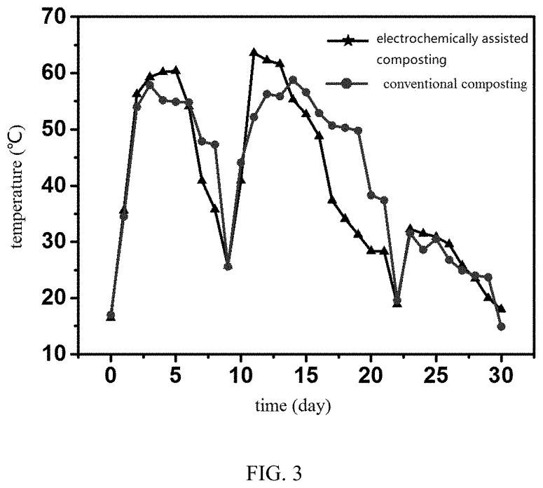

FIG. 3 is a graph showing changes in compost temperature with the turning compost time in embodiment 2 and comparative example 2. As can be seen from FIG. 3, the maximum temperature of electrochemically assisted composting is 63.3° C., the temperature of conventional composting is 58.1° C., and the temperature of electrochemically assisted composting is 5.2° C. higher than that of conventional composting. The germination index (GI) of the seed product in conventional compost is 92%, and the germination index (GI) of electrochemically assisted compost is 142%, which is 50% higher than that of conventional compost.

### 3. Composting Effect of Embodiment 3 and Comparative Example 3

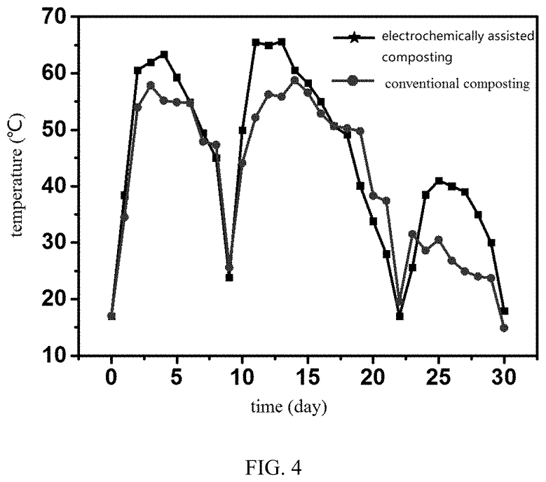

FIG. 4 is a graph showing changes in compost temperature with the turning compost time in embodiment 3 and comparative example 3. As can be seen from FIG. 4, the maximum temperature of electrochemically assisted composting is 66° C., the temperature of conventional composting is 59° C., and the temperature of electrochemically assisted composting is 7° C. higher than that of conventional composting. The germination index (GI) of the seed product in conventional compost is 98%, and the germination index (GI) of electrochemically assisted compost is 145%, which is 47% higher than that of conventional compost.

It can be seen from the above embodiments that the temperature of the electrochemically assisted composting is higher than that of conventional composting. In the second and third cycles of compost turning, the temperature difference is more significant. Under the second turning cycle, the temperature of conventional compost decreased to 55° C., while under electrochemical action, the temperature of the second cycle did not decrease, but, instead, increased by 3-4° C. relative to that of the first cycle. This is because under the action of electrochemistry, the electric energy microorganism can be enriched, and the organic matters that cannot be degraded in the process of conventional composting are utilized by the electric energy microorganisms so that the composting temperature increases. These results show that the electrochemically assisted composting can promote the redox reaction in the compost pile, improve the composting temperature rapidly, shorten the composting cycle, improve the compost maturity and save costs.

1. A device for electrochemically assisted aerobic composting of organic solid waste, comprising a compost bioreactor, a working electrode, an auxiliary electrode, and a reference electrode; the working electrode is provided on an inner wall of the compost bioreactor, and the reference electrode is situated between the working electrode and the auxiliary electrode; a ventilation pipeline with an aeration head is provided at a bottom of the compost bioreactor.

2. The device for electrochemically assisted aerobic composting of organic solid waste according to claim 1, wherein a distance between the working electrode and the auxiliary electrode is 10 cm to 100 cm.

3. The device for electrochemically assisted aerobic composting of organic solid waste according to claim 1, wherein the working electrode is at least one selected from the group consisting of a stainless steel plate, a graphite plate, a carbon felt, and a carbon cloth electrode; the auxiliary electrode is at least one selected from the group consisting of a carbon felt, a carbon cloth, and a graphite electrode; the reference electrode is one selected from the group consisting of a Hg/HgO electrode, an Ag/AgCl electrode, a hydrogen standard electrode, and a saturated calomel electrode.

4. The device for electrochemically assisted aerobic composting of organic solid waste according to claim 3, wherein the potential of the working electrode relative to the reference electrode is set to −5 V to 5 V.

5. The device for electrochemically assisted aerobic composting of organic solid waste according to claim 1, wherein the working parameters of aeration of the ventilation pipeline are: once to twice a day, each aeration is for 15 minutes to 30 minutes, a flow rate of the aeration is 0.1 L/min·m³ to 0.2 L/min·m³.

6. The device for electrochemically assisted aerobic composting of organic solid waste according to claim 1, wherein the compost bioreactor is covered with a thermal insulation layer.

7. A method for electrochemically assisted aerobic composting of organic solid waste, comprising
1) mixing organic solid waste and auxiliary material, adding water and stirring evenly to obtain compost raw material;
2) placing the composting raw material into the device of claim 1 to perform composting.

8. The method for electrochemically assisted aerobic composting of organic solid waste according to claim 7, wherein in step 1), a mass ratio of the organic solid waste and the auxiliary material is 10:(1 to 3).

9. The method for electrochemically assisted aerobic composting of organic solid waste according to claim 7, wherein in step 1), the water content of the compost raw material is 50 wt % to 60 wt %.
